(19) United States
(12) Patent Application Publication
(10) Pub. No.: US 2022/0015987 A1
(43) Pub. Date: Jan. 20, 2022

(54) Treatment Devices and Methods of Use

(71) Applicant: Filip Sedic, Stockholm (SE)

(72) Inventor: Filip Sedic, Stockholm (SE)

(21) Appl. No.: 17/325,589

(22) Filed: May 20, 2021

Related U.S. Application Data
(60) Provisional application No. 63/053,092, filed on Jul. 17, 2020.

(51) Int. Cl. A61H 23/02

(52) U.S. Cl. CPC A61H 23/02; A61H 2201/10; A61H 2201/0188; A61H 2201/0153

(57) ABSTRACT

An example treatment device includes a main body having a first end, a second end, an intermediate portion, a light source, a motor, a power source, a set of pins, and a user control. The treatment device may be configured provide a user with one or more of: electronic muscle stimulation, radiofrequency stimulation, LED light stimulation, and/or vibrational stimulation.

### FIELD

The disclosure relates generally to the field of treatment devices and methods of use. More particularly, the disclosure relates to treatment devices suitable for use on the human face and body and various methods of using the same.

### BACKGROUND

Health and appearance are important aspects of virtually all beauty regimens. As a person ages, his or her skin tends to develop wrinkles, sag, become rough, and/or develop various spots or marks. It is known that stimulating skin cells, muscles, and tissues through various means such as through light therapy, vibration therapy, electronic muscle stimulation, and/or radiofrequency stimulation can produce positive effects on a user's skin and body health. Such positive effects, for example, may include reduction of acne, production of collagen, and generally improved skin appearance. Radiofrequency stimulation applied to the skin and muscles and tissues disposed beneath the skin, for example, may tighten skin, increase muscle tone, reduce wrinkles, reduce discoloration, and/or promote the formation of new collagen or elastin.

Several devices exist which may provide light therapy, vibration therapy, electronic muscle stimulation, or radiofrequency stimulation to a user. Such devices, however, typically provide only one or two of said treatments.

A need exists, therefore, for improved treatment devices and methods of use that provide multiple treatments to a user (i.e., one or more of light, vibration, electronic muscle stimulation, and/or radiofrequency stimulation) which may be used at home quickly and efficiently.

### BRIEF SUMMARY OF SELECTED EXAMPLES

Various example treatment devices and methods of use are described.

An example treatment device comprises a main body having a first end, a second end substantially opposite the first end, and an intermediate portion extending from the first end to the second end, the second end including a base, a light source disposed within the main body, the light source configured to emit light adjacent the base, a motor disposed within the main body, the motor configured to produce pulsations of said treatment device, a set of pins attached to the base, the set of pins configured to transmit radiofrequency stimulation, a power source disposed within the main body, the power source configured to supply power to the set of pins, and a user control configured to operate the light source, motor, and power source.

Another example treatment device comprises a main body having a first end, a second end substantially opposite the first end, and an intermediate portion extending from the first end to the second end, the second end including a base, a light source disposed within the main body, the light source configured to emit light adjacent the base, a motor disposed within the main body, the motor configured to produce pulsations of said treatment device, a set of pins attached to the base, the set of pins configured to transmit radiofrequency stimulation and electronic muscle stimulation, a power source disposed within the main body, the power source configured to supply power to the set of pins, and a user control configured to operate the light source, motor, and power source.

Another example treatment device comprises a main body having a first end, a second end substantially opposite the first end, and an intermediate portion extending from the first end to the second end, the second end including a base, a light source disposed within the main body, the light source configured to emit light adjacent the base, a motor disposed within the main body, the motor configured to produce pulsations of said treatment device, a set of pins attached to the base, the set of pins configured to transmit radiofrequency stimulation and electronic muscle stimulation, the set of pins including five pins, the five pins including four similarly-shaped pins disposed around a center pin, the center pin configured to transmit radiofrequency stimulation but not electronic muscle stimulation, a power source disposed within the main body, the power source configured to supply power to the set of pins, and a user control configured to operate the light source, motor, and power source.

Additional understanding of claimed devices and methods may be obtained by reviewing the detailed description of selected examples, below, with reference to the appended drawings.

### DESCRIPTION OF FIGURES

FIG. 1 is a perspective view of a first example treatment device.

FIG. 2 is an end view of the treatment device illustrated in FIG. 1.

FIG. 3 is a side view of the treatment device illustrated in FIG. 1.

FIG. 4 is a sectional view of the treatment device illustrated in FIG. 3, taken along line 4-4.

FIG. 5 is another side view of the treatment device illustrated in FIG. 1.

FIG. 6 is another end view of the treatment device illustrated in FIG. 1.

FIG. 7 is a top view of the treatment device illustrated in FIG. 1.

FIG. 8 is a bottom view of the treatment device illustrated in FIG. 1.

FIG. 9 is a diagram illustrating components of a networked treatment device in an example embodiment.

FIG. 10 is a perspective view of a second example treatment device.

FIG. 11 is an end view of the treatment device illustrated in FIG. 10.

FIG. 12 is a side view of the treatment device illustrated in FIG. 10.

FIG. 13 is a sectional view of the treatment device illustrated in FIG. 12, taken along line 13-13.

FIG. 14 is another side view of the treatment device illustrated in FIG. 10.

FIG. 15 is another end view of the treatment device illustrated in FIG. 10.

FIG. 16 is a top view of the treatment device illustrated in FIG. 10.

FIG. 17 is a bottom view of the treatment device illustrated in FIG. 10.

FIG. 18 is a flowchart representation of an example method of using a treatment device.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF SELECTED EXAMPLES

The following detailed description and the appended drawings describe and illustrate various treatment devices and methods of use. The description and drawings are provided to enable one skilled in the art to make and use one or more example treatment devices. They are not intended to limit the scope of the claims in any manner.

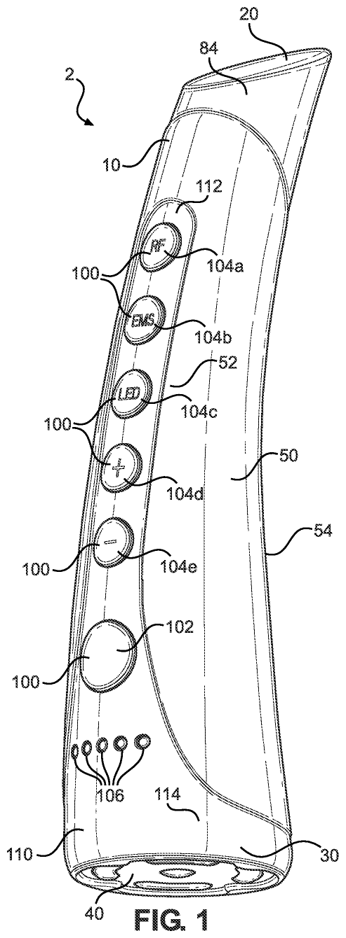

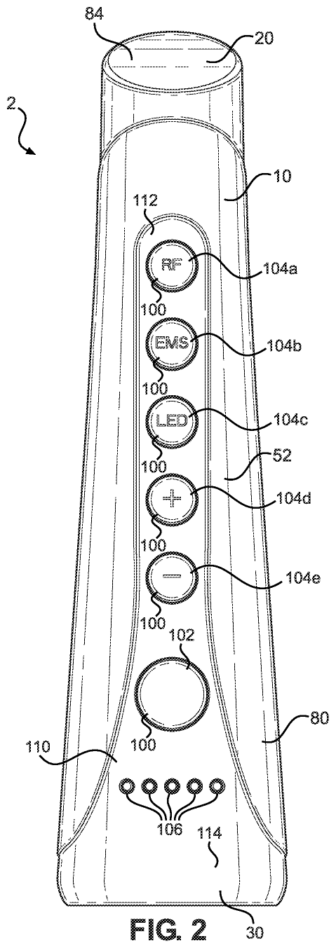

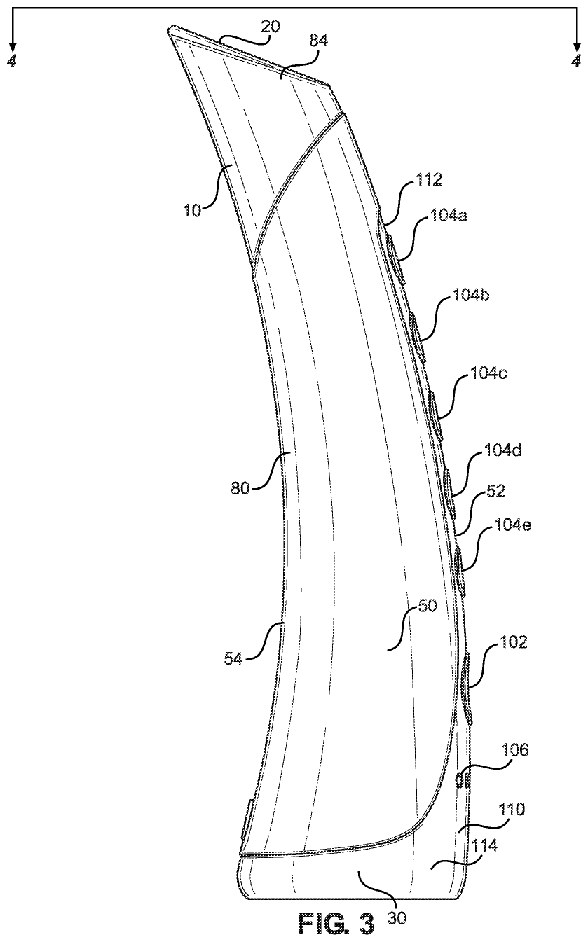

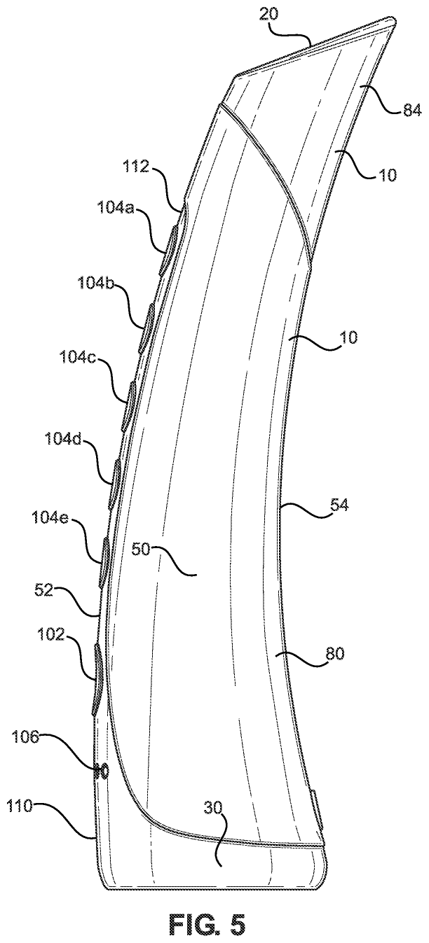

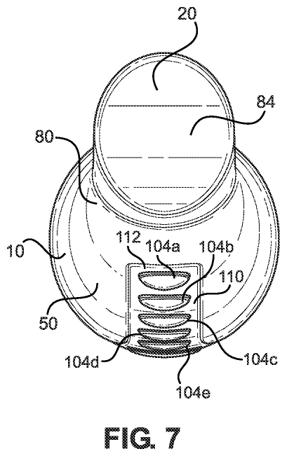

FIGS. 1, 2, 3, 4, 5, 6, 7, 8, and 9 illustrate an example treatment device 2. The treatment device 2 comprises a main body 10 that includes and/or houses several components (described in greater detail below).

The main body 10 includes at least a first end 20, a second end 30 substantially opposite the first end 20, and an intermediate portion 50 extending from the first end 20 to the second end 30. The main body 10 is substantially elongate in shape and curves slightly from the second end 30 to the first end 20. This allows for a user to easily grasp the device in his or her hand along the rear side 54 (which is disposed substantially opposite the front side 52) of the intermediate portion 50 of the treatment device 2.

The second end 30 of the main body 10 also includes a base 40. The base 40 is substantially circular in shape. The base 40 includes a base surface 41 having a first area 42 and a second area 43. The base surface 41 includes surfaces of the first and second areas 42, 43, though they are not continuous in the illustrated embodiment. The first area 42 comprises one or more plastics and is configured to allow light from the light source (described in greater detail below) to penetrate the first area 42. More specifically, the first area 42 can be relatively dark in color, but not dark enough and/or opaque enough that that the aforementioned light from the light source is incapable of penetrating the first area 42.

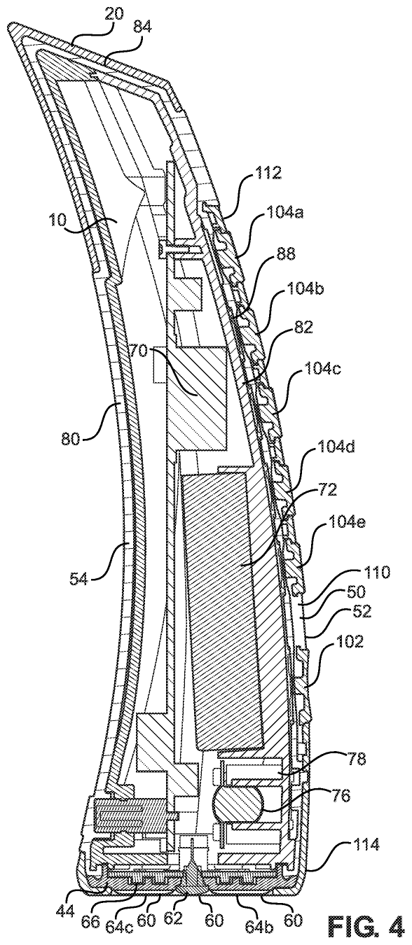

The second area 43 surrounds the first area 42 and is comprised of a metal. The first area 42 and its surface are indented relative to the surface of the second area 43. Each of the first and second areas 42, 43 of the base 40 is disposed adjacent to a base support piece 44, which is comprised of plastic and best illustrated in FIG. 4. A skilled artisan will be able to select suitable shapes and configurations of the main body according to a particular example based on various considerations including the desired shape of the main body itself and the desired functionality of the base. In some embodiments, the treatment device may not curve along its length. In other embodiments, the main body may have any shape or size, including that of a cylinder, pyramid, sphere, or any other suitable shape. In different embodiments, the base surface may include a single flat surface or may be angled or otherwise positioned in any suitable manner; it may also be made of any suitable materials, including materials similar in nature to plastic and may not include a darkened portion through which light may pass. The first and second areas may be integrally formed or comprise multiple pieces in various embodiments. The base may be adhesively or mechanically attached at the second end, or may be integrally formed with the same in different embodiments.

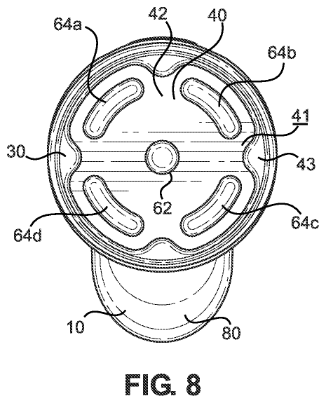

The base 40 includes a set of pins 60 which protrude from the base surface 41. As best illustrated in FIG. 8, the set of pins 60 includes a circular pin 62 and four substantially similarly shaped outer pins 64a, 64b, 64c, 64d (collectively referred to as the “pins 60”). The outer pins 64a, 64b, 64c, 64d are disposed around the circular pin 62 on the base 40. The outer pins 64a, 64b, 64c, 64d are arced in shape and are substantially the same in size. The circular pin 62 is disposed substantially at the center of the base surface 41 and substantially equidistant from the outer pins 64a, 64b, 64c, 64d. The pins 60 are comprised of a suitable metal or combination of metals in the illustrated embodiment. The pins 60 are attached to the base 40 in the illustrated embodiment via mechanical attachment mechanisms. A skilled artisan will be able to select suitable pins according to a particular example based on various considerations including the size and shape of the base, the desired functionality of the device, and any desired potential treatment locations. In other embodiments, the base may include one, two, three, four, six, or more than six pins. In different embodiments, the one or more pins may have any shape and any individual pin may be shaped the same as or differently than any other individual pin. In another embodiment, the pins may be integrally formed with the base, adhesively attached to the base, or otherwise connected to the base. In yet another embodiment, the pins may be comprised of any suitable material, including zinc or other suitable metals, which may be gold-plated (including 18 carat gold plated) or titanium plated.

The pins 60 are configured such that they can transmit electronic muscle stimulation or radiofrequency stimulation to a user. More specifically, circular pin 60 and outer pins 64a and 64c are capable of transmitting radiofrequency stimulation to a user, while the other outer pins 64b and 64d can transmit electronic muscle stimulation to a user. The pins 60 are operatively connected to the power source and second controller (described in greater detail below) such that a user may select when and whether to utilize the pins 60 for transmission of one or both types of stimulation, whether concurrently or at different times. In the illustrated embodiment, the treatment device 10 includes a second controller 66 that communicates with the first controller 70 to ensure that the pins 60 operate properly. In various other embodiments, one or more of the pins may transmit only electronic muscle stimulation and/or radiofrequency stimulation. In other embodiments, a treatment device may have one or more than two controllers. In another embodiment, the electronic muscle stimulation may be emitted in pulsations when the device is in use. When in use, the circular pin 62 and outer pins 64a and 64c produce radiofrequency stimulation that may be applied to the skin and muscles and tissues disposed beneath the skin. This allows the radiofrequency stimulation to provide one or more of the benefits described in paragraph [0002]. A skilled artisan will be able to determine the number, size, shape, and configuration of the pins and will be able to suitably determine which pins should emit electronic muscle stimulation or radiofrequency stimulation according to a particular example based on various considerations, including the desired functionality of the device and the size and shape of the same. In various embodiments, the device may include one, two, three, four, six, or more than six pins. In other embodiments, the pins may be configured in any manner relative to one another and may have any size and/or shape. In an additional embodiment, the frequency of the radiofrequency stimulation emitted by the device may be between about 0.5 megahertz (“MHz”) and about 5 MHz, between about 1 MHz and about 4 MHz, and between about 1.5 MHz and about 2 MHz. In an additional embodiment, the electronic muscle stimulation emitted by the device may be between about 10 Hz and about 200 Hz, between about 25 Hz and 100 Hz, and between about 40 Hz and about 50 Hz. In various embodiments, the device may emit electronic muscle stimulation pulses; the device may emit between about 10 pulses per second (“pps”) and 200 pps, between about 50 pps and about 150 pps, and between about 100 pps and about 110 pps.

In another embodiment, each of the pins contains a mechanism (not illustrated in the Figures) that allows for the pins to alternate between transmission of electronic muscle stimulation and radiofrequency stimulation. This mechanism is a physical mechanism that communicates with the second controller in the illustrated embodiment; however, in other embodiments, it may be an electronic mechanism that communicates with one or both controllers, a software messaging system that ensures smooth alternating between the two modes, and/or any other similar mechanism.

The second controller 66 also comprises a light source. More specifically, the light source is integrally formed with and/or attached to the controller such that the it emits visible light. The light source is comprised of light-emitting diode lights (“LED lights”) which are configured to emit one or more of blue light, red light, and/or green light. The emission of these various types of light onto human skin has known benefits. Blue LED light, for example, is known to aid in the destruction of bacteria. Red LED light is known to improve skin texture and tone, stimulate collagen, and accelerate the healing of the skin. Green LED light is known to help reduce skin discoloration and tighten the skin. These and other LED light colors have other known benefits, as well. The controller 66 is configured such that the light emitted by the light source penetrates through the base and onto a user, when in use. A skilled artisan will be able to select a suitable light source for a particular device according to a particular example based on various considerations, including the size and shape of the device and the type(s) of light to be emitted by the light source. In another example, the light source may only emit one or more of blue, red, or green light. In an alternative example, the light source may also emit one or more of white, black, yellow, purple, pink, cyan, and orange light. In other embodiments, the light source may be comprised of any type of light, rather than an LED light. In various embodiments, the light source may comprise one, two, three, four, five, six, seven, eight, or more than eight individual lights. In different embodiments, the light source may be separate from the second controller or any other component via a physical attachment or an adhesive; alternatively, various lights may be disposed on multiple other components via any suitable mechanism. In some embodiments, an individual light may only emit a single color; in other embodiments, an individual light may emit one, two, three, four, or more than four colors. Additionally, the lights may have different shapes in different embodiments, including box, cylinder, pyramid, sphere, and other suitable shapes.

The first controller 70 and the second controller 66 in the illustrated embodiment include a printed circuit board assembly (“PCBA”) and related circuitry and a printed circuit board (“PCB”), respectively; however, in other embodiments, either controller may comprise any device suitable to control the device's components. Moreover, one or both of the first and second controllers 70, 66 may be operatively connected to an interface 90, allowing one or both to communicate with a second device (described in greater detail below).

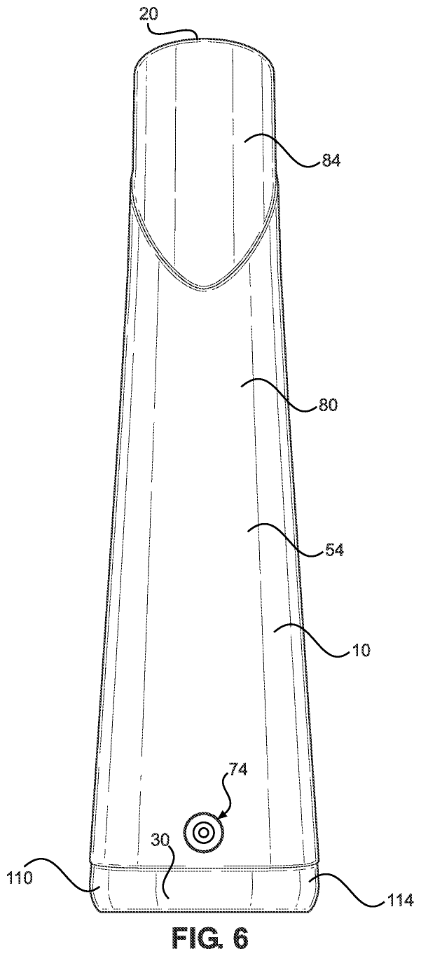

The main body 10 also houses a power source 72. The power source 72 is operatively connected to and provides power to the various components of the treatment device 2 (such as the first and second controllers 70, 66, pins 60, and others described below) so that they may function efficiently and properly. The power source 72 comprises a rechargeable lithium-ion battery in the illustrated embodiment and may be charged through a charging port, such as DC jack 74 best shown in FIG. 6. The power source 72 is securely placed within the main body 10. A skilled artisan will be able to select a suitable power source according to a particular example based on various considerations, including the various components housed within the device and the desired dimensions of the device. In other embodiments, the power source may comprise one, two, three, or more than three replaceable dry cell batteries. In different embodiments, the power source may comprise a lithium polymer battery or batteries.

A motor 76 is also housed within the main body 10. It is operatively connected to the power source 72 and the first controller 70. The motor 76 is at least partially disposed within in a frame 78 disposed within the main body 10. The frame 78 is configured to support the motor 76 and keep the motor 76 secured at a particular location inside the main body 10, as well as provide general structural support to the treatment device 2. Specifically, the frame 78 includes an arm that defines a slot into which the motor 76 is disposed. The motor 76 is disposed substantially adjacent the second side 30 so its vibrations may be efficiently transmitted to the user via the base 40.

The first controller 70 provides instructions to the motor 76, which is powered by the power source 60. A high or low-frequency motor 76 may be used to create pulsations that vibrate the treatment device in various embodiments. The motor 76 in this embodiment is configured to produce a range of frequencies that may provide beneficial treatment of the skin. When the treatment device 2 is in operation, and the base 40 in particular, is applied to the body, such as the face or neck, the motor 76 provides vibrations to the skin. A skilled artisan will be able to select suitable motors, frames, and vibration ranges based on various considerations, including the size and shape of the treatment device and the desired vibration strength of the device. Examples of suitable ranges for vibration frequencies includes vibration frequencies between about 80 Hertz (“Hz”) and about 200 Hz, vibration frequencies between about 100 Hz and about 180 Hz, and vibration frequencies between about 120 Hz and about 150 Hz. In another embodiment, the device may include two or more motors. In another embodiment, a motor may be stochastic. In other embodiments, the frame may be omitted. In different embodiments, the frame may not include a slot or arm, but may instead house the device entirely within its main body. In some embodiments, there may be more than one motor; in embodiments containing multiple motors, the motors may vary from one another in frequency output. In other embodiments, the motor may be controlled by the second controller.

The first controller 70 also controls an interface 90 that is a component of the first controller 70. The interface 90 allows the treatment device 2 to communicate with a second device, such as a personal computer, tablet, mobile telephone, or other electronic device (not illustrated in the Figures). Using the interface 90, the treatment device 2 can send information to other devices so that the other device(s) may collect data pertaining to the use of the treatment device 2. Furthermore, the treatment device 2 may receive control signals from another device that can indicate that the treatment device 2 should turn on or off, increase or decrease speed, switch to a different vibration, lighting, heating or cooling pattern, and/or switch to a pre-set pattern desired by the user or recommended by the other device, among other instructions. The interface 90 can be a wired or a wireless interface, such as a wireless transceiver that transmits control signals between the treatment device 2 and the second device. A skilled artisan will be able to select a suitable interface based on various considerations, including the device with which the treatment device will communicate and the size and shape of the main body. In some embodiments, the interface is a radiofrequency (“RF”) transceiver used to transmit and receive RF signals between the treatment device and other devices. One example of an RF transceiver that could be used is a low power 2.4 GHz RF transceiver. In various embodiments, the treatment device may also include antennas for transmitting and receiving signals between the treatment device and other devices. In such examples, the interface can use BLUETOOTH®, Wi-Fi, infrared, laser light, visible light, acoustic energy, or one of several other methods to transmit information wirelessly between the treatment device and another device. In another embodiment, the first controller can specifically communicate with another device to confirm the treatment device's authenticity. In different embodiments, the second controller may also communicate with the second device.

In some embodiments, the treatment device is connected to a network via the second device. In other embodiments, the treatment device is directly connected to a wireless router or cellular phone network and may connect with the second device in any of said manners. Accordingly, the treatment device can be controlled via personal computer, tablet, mobile phone, or other suitable electronic devices a user using the personal computer, tablet, phone, or other device. An application on a mobile phone, for instance, may communicate with the treatment device in order to control the treatment device's functionality.

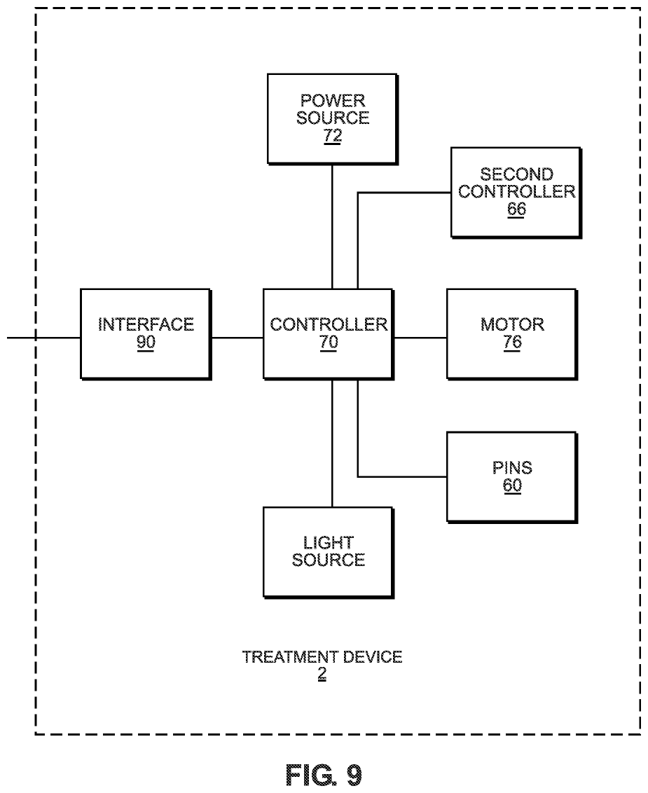

FIG. 9 is a diagram illustrating components of a networked treatment device, such as treatment device 2, in accordance with an example embodiment. In this embodiment, the treatment device includes a controller, such as first controller 70, a motor, such as motor 76, a light source, such as the light source described above, a power source, such as power source 72, a second controller, such as second controller 66, a set of pins, such as pins 60, and an interface, such as interface 90. As explained above, the treatment device can be connected to a network via a personal computer, tablet, mobile telephone, or other electronic device or can be directly connected to a wireless router or cellular phone network. Thus, the treatment device can be controlled by, transmit data to, and/or receive data from the personal computer, tablet, mobile telephone, or other electronic device via the aforementioned mechanisms. The interface may be wired or wireless and may include any of those described above. A skilled artisan will be able to determine how to suitably connect the treatment device with other devices according to a particular example based on various considerations, including the desirability of doing so and the devices to which connection would be beneficial. In some embodiments, the treatment device may not include an interface and, thus, may not communicate with other devices. In different embodiments, the treatment device may only transmit data to other devices; it may not receive any data and cannot be controlled via said other devices in this embodiment.

Example data that the treatment device may communicate to one or more of a personal computer, tablet, mobile telephone, or other electronic device may include the number of uses of the treatment device, the durations of the various uses of the treatment device, the user's preferred treatment device settings, and various other types of information related to the use of the device.

The main body 10 also includes a silicone cover 80 that extends along the intermediate portion 50 such that it is adjacent to the first end 20 and the second end 30. The silicone cover 80 is adjacent a shell 82 and an endpiece 84 disposed adjacent the first end 20; the silicone cover 80, shell 82, endpiece 84, and base 40 cooperatively provide the main body 10 with its overall shape and structure. The shell 82 and endpiece 84 are comprised of plastic in the illustrated embodiment. The silicone cover 80 is designed to be contacted by a user when the treatment device 2 is in use. The silicone cover 80 also defined an indented portion (not illustrated in the Figures) that allows for a user control, a thin film switch 88, and its related circuitry (described in greater detail below) to be placed within the indented portion and secured in place by a securing piece (described in greater detail below). In the illustrated embodiment, the indented portion defines a set of grooves (not illustrated in the Figures) that are configured to align with various portions of the user control, thin film switch 88, and securing piece. A skilled artisan will be able to select a suitable silicone cover, shell, and endpiece according to a particular example based on various considerations, including the desired shape and size of the treatment device and the desired functionalities of the same. In another embodiment, the silicone cover may be omitted. In various embodiments, the shell may be comprised of one, two, three or more than three pieces and the pieces may be secured to one another via one or more adhesives or attachment mechanism; one or more pieces may also be integrally formed. The shell and endpiece may be attached to one another via any suitable mechanism, including form-fitting, adhesives, and/or physical attachment mechanisms. The silicone cover may be attached to the shell via adhesives, attachment mechanisms, or form-fitting in various embodiments. In some embodiments, the silicone may be medical-grade and/or a specially formulated, ultra-hygienic silicone. In different embodiments, the shell, endpiece, and/or silicone cover may have any suitable shape. The endpiece may also contain a logo or other marking in some embodiments.

The user control 100 is disposed over the thin film switch 90 comprises a number of buttons that may be used to power on and off the treatment device 2 and communicate a user's desired functionality to the first and second controllers 70, 66 once the treatment device 2 has been powered on. The thin film switch 88 is disposed between the shell 82 and the silicone cover 80 such that a user pressing the user control 100 will initiate a desired function via cooperation between the user control 100, shell 82, thin film switch 88, and silicone covering 80. The thin film switch 88 comprises a capacitive sensor. In the illustrated embodiment, the user control 100 comprises a total of six buttons, including the power button 102 and a set of operation buttons 104a, 104b, 104c, 104d, 104e. The power button 102 is disposed closer to the base 40 than are the operation buttons 104a, 104b, 104c, 104d, 104e and, when pressed, powers on and off the treatment device 2. Each of the buttons of the user control 100 are substantially circular in shape; the operation buttons 104a, 104b, 104c, 104d, 104e are substantially identical in diameter, each diameter being smaller than that of the power button 102. In the illustrated embodiment, operation button 104a controls the emission of radiofrequency stimulation via the base 40, operation button 104b controls the emission of electronic muscle stimulation via the base 40, and operation button 104c controls the emission of light via the light source via the base 40. Operation button 104d increases the intensity of one or more of the above emissions, while operation button 104e decreases the intensity of one or more of the above emissions. The motor 76, for example, may emit vibrations while the treatment device 2 in emitting radiofrequency stimulation and/or electronic muscle stimulation. The motor 76 can increase and decrease its output based upon a user's manipulation of the user control 100 while the treatment device 2 emits one or more other types of stimulation. The user control 100 is disposed on the front side 52 of the intermediate portion 50 so that a user may easily press one of more of the buttons with his or her finger while using the treatment device 2 and placing the base 40 adjacent a desired treatment area. More specifically, the user control 100 is disposed adjacent the silicone cover 80 and thin film switch 88 and positioned such that its buttons are aligned over the grooves defined by the indented portion of the silicone cover 80. The user control 100 is held in place by a securing piece (described in greater detail below) in the illustrated embodiment. The user control 100 is comprised of plastic in the illustrated embodiment. A skilled artisan will be able to select a suitable user control according to a particular example based on various considerations, including the size and shape of the silicone cover and the desired functionality of the device. In various embodiments, the user control may have one, two, three, four, five, or more than six buttons (any functionality of the device may be controlled by any of the buttons) and the buttons may have any suitable size and/or shape. In some embodiments, one button may control more than one function of the device. In another embodiment, the power button may be disposed on the rear side of the device. The user control may be comprised of other suitable materials, such as one or more metals, in different embodiments. In a different embodiment, zero, two, or more than two thin film switches may be used; in others, any suitable mechanism may be used in place of a thin film switch.

The securing piece 110 is comprised of plastic and disposed adjacent to the user control 100 and silicone cover 80 of the main body 10. The securing piece 110 widens along its length such that is wider at its first end 112 than at its second end 114. Most of the securing piece 110 is disposed on the front side 52 of the intermediate portion, though it does encircle the second end 30 of the main body 10 such that it is adjacent to the base 40. The securing piece 110 defines a first set of passageways through which the buttons of the user control 110 extend and a second set of passageways that is aligned with the passageways of the silicone cover (the passageways are not illustrated in the Figures). The first set of passageways allows for the user to interact with the user control 100; the second set of passageways allow for light from an indicator 106 to be emitted through the second set of passageways. The light emitted via these passageways provides a user with particular information, such as whether the treatment device 2 is powered on, the strength or intensity of one or more of the functionalities of the treatment device 2, the particular functionality currently in use, and/or the treatment device's 2 power level, which may indicate that the treatment device 2 may need to be recharged shortly. Each passageway of the first and second set of passageways are cylindrical in shape and define two circular openings in the illustrated embodiment; there are six passageways in the first set of passageways and five passageways in the second set of passageways, as well. A skilled artisan will be able to select a suitable securing piece according to a particular example based on various considerations, including the desired functionalities of the device and the size and shape of the same. In various embodiments, the securing piece may comprise one, two, three, more than three sets of passageways, each including any number of individual passageways which may be sized, shaped, and aligned in any suitable fashion. In a different embodiment, the securing piece may be comprised of any suitable material, including various metals. In other embodiments, the securing piece may cover any portion of the silicone cover, base, shell, endpiece, and/or other component.

In use, a user may grasp the main body 10 of the treatment device 2 in his or her hand, turn the treatment device 2 on via the user control 100, and place the base 40 adjacent to a desired treatment area. The user will then select a desired functionality by pressing one or more of the operation buttons 104a, 104b, 104c, 104d, 104e. The user may also select a functionality prior to placing the treatment device 2 against his or her skin. The treatment device 2 will then perform the function selected by the user. At this time, the user may increase or decrease the intensity of the function via the user control 100. Subsequently, the user will be able to utilize the same function again, choose and implement a second function, and/or power off the skin treatment device 2.

Once a treatment has been selected by a user, the treatment device 2 will do one or more of providing electronic muscle stimulation, radiofrequency stimulation, vibration and/or pulsations, and/or light therapy. The treatment device 2 may perform these treatments in any order, for any duration, and through contact with or placement near the skin via various portions of the device and as described above. One or more treatments may be emitted at the same time.

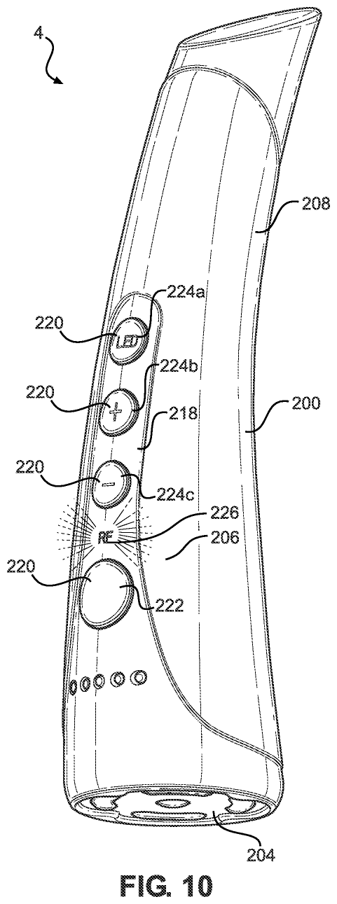

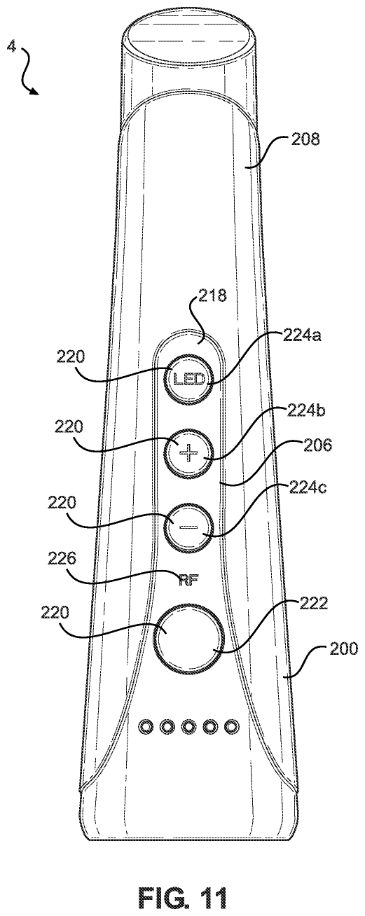

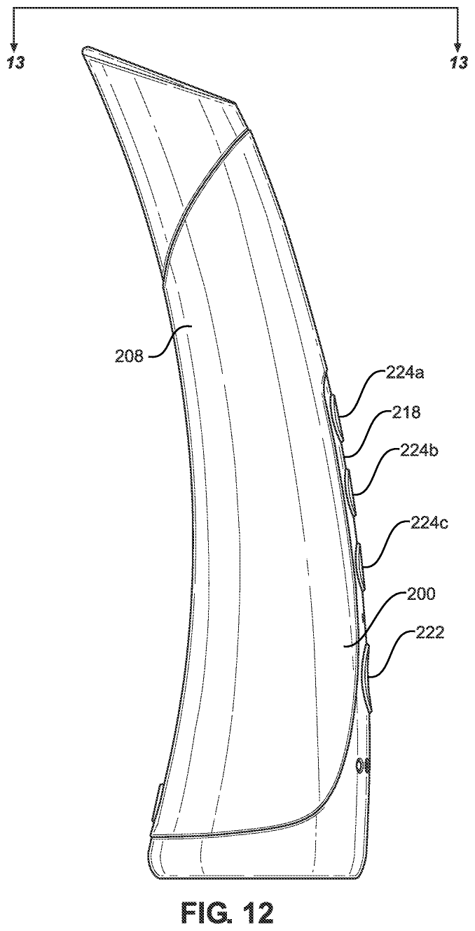

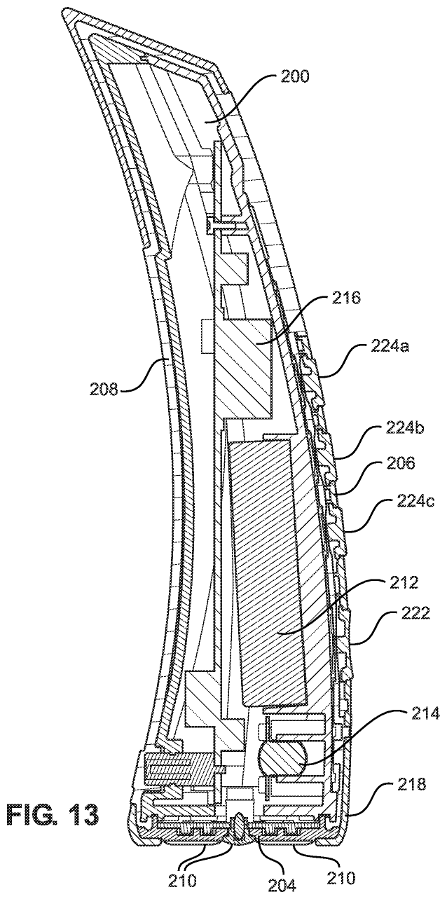

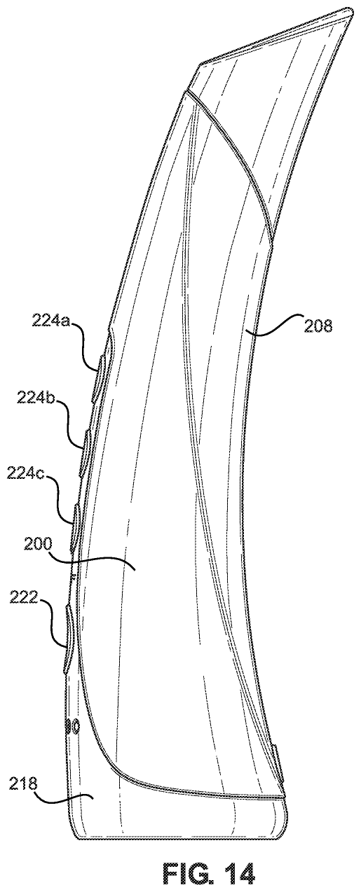

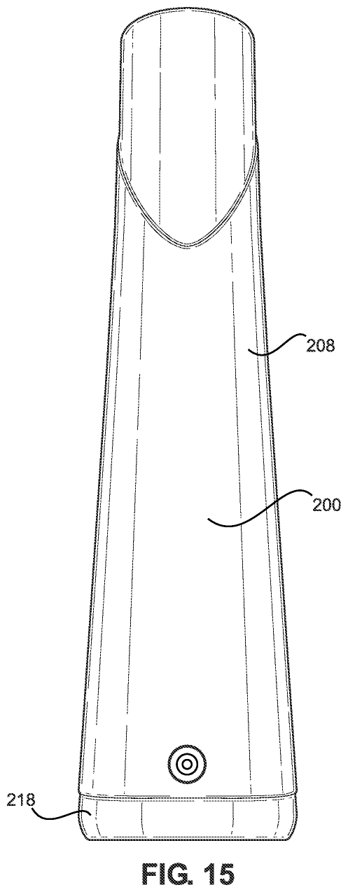

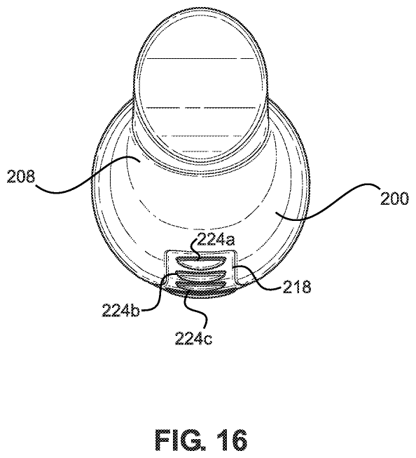

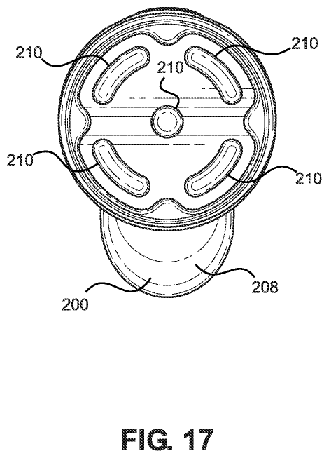

FIGS. 10, 11, 12, 13, 14, 15, 16, and 17 illustrate another example treatment device 4. The treatment device 4 is similar to the treatment device 2 illustrated in FIGS. 1 through 9, except as described below. Thus, the skincare device 4 comprises at least a main body 200, a base 204, an intermediate portion 206, a silicone cover 208, a set of pins 210, a power source 212, a motor 214, a controller 216, a securing piece 218, and a user control 220.

Treatment device 4 can produce radiofrequency stimulation; however, unlike treatment device 2, it does not produce electronic muscle stimulation. Accordingly, the power source 212 and the set of pins 210 do not cooperatively act to produce electronic muscle stimulation.

The user control 220, thus, has fewer buttons than the user control 100 illustrated in the first embodiment. The user control 220 instead includes a power button 222 and a set of operation buttons 224a, 224b, 224c. Operation button 224a controls the emission of LED light via the light source, operation button 224b increases the intensity of the selected function, operation button 224c decreases the intensity of the selected function, and power button 222 powers on and off the treatment device 4. In other embodiments, greater or fewer than four buttons may be included, and they may be aligned in any way.

Pressing the user control 220 also begins the radiofrequency stimulation. At such a time, a radiofrequency stimulation indicator 226 will be will light up to indicate that radiofrequency stimulation is being emitted by the treatment device 4.

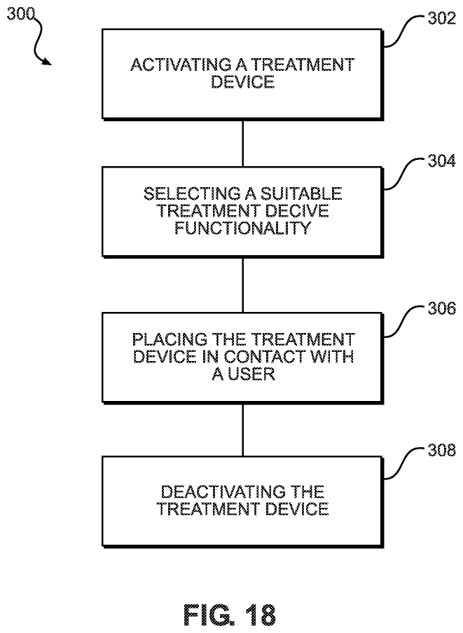

FIG. 18 is a flowchart representation of an example method 300 of using a treatment device. Performance of this method results in the treatment of the user's body, such as the face and/or neck, by a treatment device. This method can be used for treating any portion of a human body.

An initial step 302 comprises activating a treatment device, such as treatment device 2 or 4 by pressing a user control, such as user control 100 or 220, to ready the treatment device for use.

Another step 304 comprises selecting a suitable treatment functionality, such as electronic muscle stimulation, radiofrequency stimulation, light stimulation, and/or vibration stimulation, or any other suitable treatment. A treatment pattern may be selected via the user control. Selection of a particular treatment pattern may be communicated to a user via a light source, such as indicator 106, which may emit different patterns, colors, or flashes of light to indicate the various treatment patterns.

Another step 306 comprises placing the treatment device in contact with or adjacent the user's preferred treatment area. Doing so allows the treatment device to treat the skin via the selected treatment functionality.

Another step 308 comprises deactivating the treatment device. Deactivation of the treatment device may occur automatically at the end of a pre-set treatment or via manipulation of the user control.

It is noted that it is advantageous to complete this method 300 in the order illustrated and described. However, any order is considered suitable.

In all examples, a treatment device and its various components may be formed of any suitable material, including presently known and later-developed materials. A skilled artisan will be able to select appropriate materials for an example treatment device based on various considerations, including the size and shape of the treatment device, the motor housed within the treatment device, the light source housed within the treatment device, and the particular electronic muscle and/or radiofrequency stimulation desired.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A treatment device, comprising:
a main body having a first end, a second end substantially opposite the first end, and an intermediate portion extending from the first end to the second end, the second end including a base;
a light source disposed within the main body, the light source configured to emit light adjacent the base;
a motor disposed within the main body, the motor configured to produce pulsations of said treatment device;
a set of pins attached to the base, the set of pins configured to transmit radiofrequency stimulation;
a power source disposed within the main body, the power source configured to supply power to the set of pins; and
a user control configured to operate the light source, motor, and power source.

2. The treatment device of claim 1, wherein the light source comprises light-emitting diode (“LED”) lights.

3. The treatment device of claim 2, wherein the light source is configured to emit red light.

4. The treatment device of claim 3, wherein the light source is configured to emit green light.

5. The treatment device of claim 4, wherein the light source is configured to emit blue light.

6. The treatment device of claim 5, wherein the main body includes a silicone cover extending along the intermediate portion.

7. The treatment device of claim 6, wherein the set of pins includes five pins.

8. The treatment device of claim 7, wherein the five pins of the set of pins includes four pins disposed around a center pin.

9. The treatment device of claim 8, wherein the five pins are comprised of zinc.

10. The treatment device of claim 9, wherein the set of pins transmits radiofrequency stimulation at approximately 2 MHz.

11. The treatment device of claim 10, wherein the base defines a base surface comprised of plastic; and
wherein the base surface allows the light emitted by the light source to be transmitted through the base surface.

12. A treatment device, comprising:
a main body having a first end, a second end substantially opposite the first end, and an intermediate portion extending from the first end to the second end, the second end including a base;
a light source disposed within the main body, the light source configured to emit light adjacent the base;
a motor disposed within the main body, the motor configured to produce pulsations of said treatment device;
a set of pins attached to the base, the set of pins configured to transmit radiofrequency stimulation and electronic muscle stimulation;
a power source disposed within the main body, the power source configured to supply power to the set of pins; and
a user control configured to operate the light source, motor, and power source.

13. The treatment device of claim 12, wherein the set of pins transmits radiofrequency stimulation between about 1 MHz and about 5 MHz.

14. The treatment device of claim 13, wherein the set of pins transmits electronic muscle stimulation between about 40 Hz and about 50 Hz.

15. The treatment device of claim 14, wherein the main body includes a silicone cover extending along the intermediate portion.

16. The treatment device of claim 15, wherein the motor produces pulsations between about 80 Hz and about 200 Hz.

17. The treatment device of claim 16, wherein the set of pins includes five pins.

18. The treatment device of claim 17, wherein the five pins of the set of pins includes four pins disposed around a center pin.

19. The treatment device of claim 18, wherein the five pins are comprised of zinc.

20. A treatment device, comprising:
a main body having a first end, a second end substantially opposite the first end, and an intermediate portion extending from the first end to the second end, the second end including a base;
a light source disposed within the main body, the light source configured to emit light adjacent the base;
a motor disposed within the main body, the motor configured to produce pulsations of said treatment device;
a set of pins attached to the base, the set of pins configured to transmit radiofrequency stimulation and electronic muscle stimulation, the set of pins including five pins, the five pins including four similarly-shaped pins disposed around a center pin, the center pin configured to transmit radiofrequency stimulation but not electronic muscle stimulation;
a power source disposed within the main body, the power source configured to supply power to the set of pins; and
a user control configured to operate the light source, motor, and power source.
